(19) United States
(12) Patent Application Publication
(10) Pub. No.: US 2021/0241910 A1
(43) Pub. Date: Aug. 5, 2021

(54) LEARNING ASSISTANCE APPARATUS AND LEARNING ASSISTANCE METHOD

(71) Applicant: CANON MEDICAL SYSTEMS CORPORATION, Otawara-shi (JP)

(72) Inventors: Minoru NAKATSUGAWA, Yokohama (JP); Yusuke KANO, Nasushiobara (JP)

(73) Assignee: CANON MEDICAL SYSTEMS CORPORATION, Otawara-shi (JP)

(21) Appl. No.: 17/152,987

(22) Filed: Jan. 20, 2021

(30) Foreign Application Priority Data
Jan. 30, 2020 (JP) 2020-013906

(51) Int. Cl. G16H 50/20; G06N 5/04; G06N 5/02; G06N 20/00

(52) U.S. Cl. CPC G16H 50/20; G06N 20/00; G06N 5/022; G06N 5/04

(57) ABSTRACT

A learning assistance apparatus according to an embodiment includes processing circuitry. Based on a knowledge base in which a condition of medical data as an index for medical judgment and medical knowledge derived from the condition are associated with each other, and a model designed to derive a medical inference result from a condition of medical data concerning a subject in response to input of the medical data, the processing circuitry compares the medical data condition related to the derivation of the medical knowledge and the medical data condition related to the derivation of the inference result, for each item of the medical data, and outputs a result of the comparison.

### CROSS-REFERENCE TO RELATED APPLICATIONS

This application is based upon and claims the benefit of priority from Japanese Patent Application No. 2020-013906, filed on Jan. 30, 2020; the entire contents of which are incorporated herein by reference.

### FIELD

Embodiments described herein relate generally to a learning assistance apparatus and a learning assistance method.

### BACKGROUND

In medical facilities such as hospitals, diagnosis and prognosis prediction have been conventionally performed by using a knowledge base, such as clinical practice guidelines, prescribing conditions of medical data as an index for medical judgment. In recent years, models (trained models) have been created by conducting machine learning using medical data of a plurality of subjects accumulated in a medical facility.

Such a model enables derivation of diagnosis or prognosis prediction of a subject in response to input of medical data collected from the subject. Unfortunately, depending on the created model, medical data conditions related to the derivation of diagnosis or prognosis prediction can conflict with the medical data conditions prescribed in the knowledge base.

### BRIEF DESCRIPTION OF THE DRAWINGS

FIG. 1 is a diagram illustrating a configuration example of a learning assistance system according to an embodiment;

FIG. 2 is a diagram illustrating a function configuration example of a learning assistance apparatus according to the embodiment;

FIG. 3 is a schematic view illustrating an example of biological parameter comparison results according to the embodiment;

FIG. 4 is a flowchart illustrating an example of processing performed by the learning assistance apparatus of the embodiment;

FIG. 5 is a diagram illustrating a function configuration example of a learning assistance apparatus according to a first modification;

FIG. 6 is a view illustrating an example of a screen displayed by a visualization function of the first modification; and

FIG. 7 is a view illustrating another example of the screen displayed by the visualization function of the first modification.

### DETAILED DESCRIPTION

A learning assistance apparatus according to an embodiment includes processing circuitry. Based on a knowledge base in which a condition of medical data as an index for medical judgment and medical knowledge derived from the condition are associated with each other, and a model designed to derive a medical inference result from a condition of medical data concerning a subject in response to input of the medical data, the processing circuitry compares the medical data condition related to the derivation of the medical knowledge and the medical data condition related to the derivation of the inference result, for each item of the medical data, and outputs a result of the comparison.

Hereinafter, an embodiment of a learning assistance apparatus and a learning assistance method will be described with reference to the drawings.

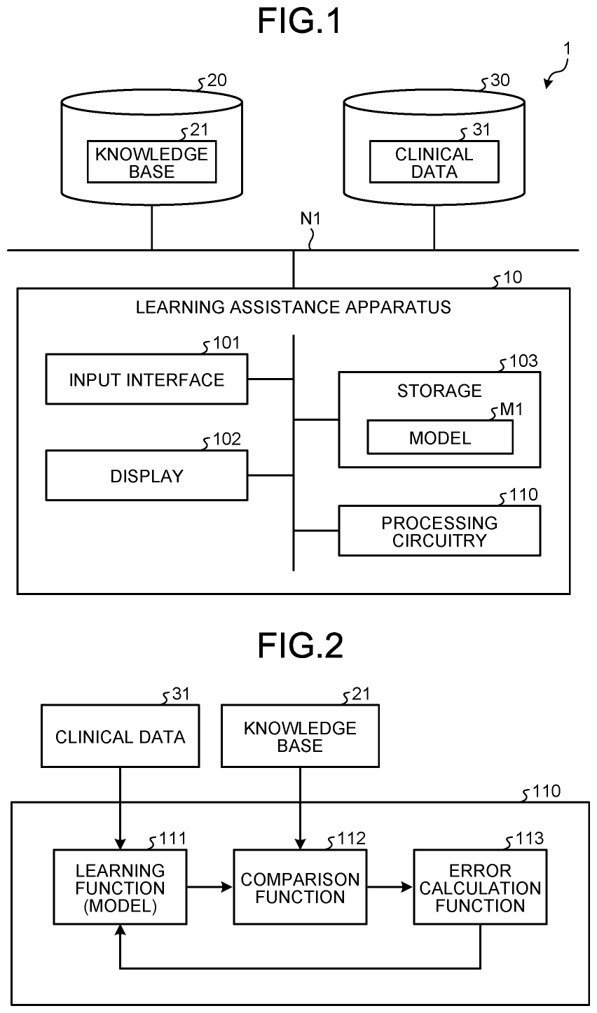

FIG. 1 is a block diagram illustrating a configuration example of a learning assistance system according to the embodiment. As illustrated in FIG. 1, a learning assistance system 1 includes a learning assistance apparatus 10, a knowledge base storage apparatus 20, and a clinical data storage apparatus 30. The learning assistance apparatus 10, the knowledge base storage apparatus 20, and the clinical data storage apparatus 30 are installed in, for example, a medical facility such as a hospital, and are connected to each other so as to allow communication via a network N1.

Note that the learning assistance apparatus 10, the knowledge base storage apparatus 20, and the clinical data storage apparatus 30 may be installed in any place that enables connection via a network. For example, the learning assistance apparatus 10 and the knowledge base storage apparatus 20 may be installed in a different place (e.g., a data center) from the medical facility where the clinical data storage apparatus 30 is installed.

The knowledge base storage apparatus 20 is a storage apparatus that stores a knowledge base 21. The knowledge base storage apparatus 20 is achieved by, for example, computer equipment such as a database (DB) server, and stores the knowledge base 21 in a storage such as a semiconductor memory including a random access memory (RAM) and a flash memory, a hard disk, and an optical disk.

The knowledge base 21 is data in which conditions of medical data as an index for medical judgment and medical knowledge derived from the conditions are associated with each other. The knowledge base 21 stores data based on guidelines, such as medical papers and clinical practice guidelines, created from a medical viewpoint. More specifically, the knowledge base 21 stores data prescribing a relation between conditions of medical data as an index (basis) for medical judgment such as diagnosis, treatment, and prognosis prediction of a disease, and medical knowledge derived from the conditions. The knowledge base storage apparatus 20 stores, for example, conditions of biological parameters as the index, and medical knowledge such as a medical risk, a disease name, and prognosis prediction derived from the conditions in association with each other, as the knowledge base 21. Examples of the biological parameters include medical data obtained by various tests, such as heart rate and blood pressure, a patient attribute such as age, gender and race, and a social attribute such as family structure. Each biological parameter condition is a set of an item and a condition value of each biological parameter used as the index. The condition value is, for example, a medical data value obtained by various tests, such as heart rate and blood pressure. The condition value may quantitatively represent a threshold or a numerical value range, or may qualitatively represent a tendency of a chronological change such as an increase and a decrease.

The clinical data storage apparatus 30 is a storage apparatus that stores clinical data 31. The clinical data storage apparatus 30 is achieved by, for example, computer equipment such as a DB server, and stores the clinical data 31 in a storage such as a semiconductor memory including a RAM and a flash memory, a hard disk, and an optical disk.

The clinical data 31 is a data group recording test results or the like conducted on subjects. For example, the clinical data storage apparatus 30 stores clinical data recording various test results conducted on each subject in chronological order in association with a patient ID identifying the subject. That is, the clinical data includes various biological parameters (medical data) collected from each subject.

In the present embodiment, the clinical data storage apparatus 30 stores the clinical data 31 used for creating a model M1 described later (hereinafter referred to as learning data), and the clinical data 31 used for verifying the model M1 (hereinafter referred to as verification data). In this case, the learning data may include not only the clinical data of each subject, but also a diagnostic result of a medical practitioner, such as a doctor, derived from this clinical data, as training data.

The learning assistance apparatus 10 executes processing related to the creation of the model M1 capable of deriving a medical inference result such as disease diagnoses, therapeutic effect determination, and prognosis prediction based on the data stored in the knowledge base storage apparatus 20 and the clinical data storage apparatus 30.

For example, the learning assistance apparatus 10 executes a process of creating the model M1 by using the learning data stored in the clinical data storage apparatus 30. The learning assistance apparatus 10 also executes a process of adjusting an operation of the model M1 based on the biological parameter conditions prescribed in the knowledge base 21 of the knowledge base storage apparatus 20. The learning assistance apparatus 10 is achieved by, for example, computer equipment such as a workstation.

As illustrated in FIG. 1, the learning assistance apparatus 10 includes an input interface 101, a display 102, a storage 103, and processing circuitry 110. The input interface 101, the display 102, the storage 103, and the processing circuitry 110 are mutually connected.

The input interface 101 receives various input operations from an operator, converts the received input operations to electrical signals, and outputs the signals to the processing circuitry 110. The input interface 101 is achieved by, for example, a mouse, a keyboard, a trackball, a switch, a button, a joystick, a touchpad that allows an operator to perform input operations by touching its operation surface, a touchscreen in which a display screen and a touchpad are integrated, a noncontact input circuit using an optical sensor, or a voice input circuit.

The input interface 101 may be also composed of a tablet terminal or the like capable of performing wireless communication with the main body of the learning assistance apparatus 10. Additionally, the input interface 101 is not limited to the one including a physical operation component such as a mouse and a keyboard. Examples of the input interface 101 also include an electrical signal processing circuit that receives electrical signals corresponding to input operations from external input equipment provided separately from the learning assistance apparatus 10 and outputs the signals to the processing circuitry 110.

The display 102 displays various information. The display 102 displays, for example, a processing result of the processing circuitry 110 under the control of the processing circuitry 110. The display 102 also displays a graphical user interface (GUI) for receiving various instructions, various settings, or the like from an operator via the input interface 101. The display 102 is, for example, a liquid crystal display or a cathode ray tube (CRT) display. The display 102 may be desktop type or may be composed of a tablet terminal or the like capable of performing wireless communication with the main body of the learning assistance apparatus 10.

The storage 103 is achieved by, for example, a semiconductor memory including a RAM and a flash memory, a hard disk, or an optical disk. The storage 103 stores, for example, computer programs for allowing the circuits included in the learning assistance apparatus 10 to achieve their functions. The storage 103 also stores, for example, various data acquired from the knowledge base storage apparatus 20 and the clinical data storage apparatus 30. Additionally, the storage 103 stores, for example, the model M1.

The processing circuitry 110 controls the entire processing of the learning assistance apparatus 10. The processing circuitry 110 executes, for example, a learning function 111, a comparison function 112, and an error calculation function 113 as illustrated in FIG. 2. The learning function 111 is an example of an adjustment unit. The comparison function 112 is an example of a comparison unit and an output unit. The error calculation function 113 is an example of an error calculation unit. FIG. 2 is a diagram illustrating a function configuration example of the learning assistance apparatus 10.

For example, the respective processing functions executed by the learning function 111, the comparison function 112, and the error calculation function 113 are recorded in the storage 103 in the form of computer-executable programs. The processing circuitry 110 is a processor that reads out each computer program from the storage 103 and executes the computer program to achieve a function corresponding to the computer program. In other words, the processing circuitry 110 that has read out each computer program has the corresponding function illustrated in the processing circuitry 110 of FIG. 2.

The learning function 111 creates the above model M1 by using the learning data stored in the clinical data storage apparatus 30. More specifically, the learning function 111 performs machine learning based on an algorithm such as logistic regression, neural networks, and deep learning by using the clinical data 31 of each subject and a doctor's judgment result (e.g., a medical judgment result such as disease diagnoses, therapeutic effect determination, and prognosis prediction) for the clinical data 31. The learning function 111 creates a trained model composed of a network such as a convolutional neural network (CNN) and a feedforward neural network (FNN), and stores the created trained model as the model M1 in the storage 103.

The model M1 is designed to derive the medical inference result such as disease diagnoses, therapeutic effect determination, and prognosis prediction in response to input of the clinical data of a subject to be diagnosed. More specifically, the learning function 111 creates the model M1 designed to output the inference result such as disease diagnoses, therapeutic effect determination, and prognosis prediction from the biological parameter conditions included in the clinical data by learning a relation between the biological parameter conditions included in the clinical data and the diagnostic result of a medical practitioner as the training data.

The above model M1 is represented by, for example, a composite function with parameters obtained by composition of a plurality of functions. The composite function with parameters is defined by a combination of a plurality of adjustable functions and parameters.

For example, when the model M1 is composed of the FNN, the composite function with parameters is defined by a combination of a linear relation between respective layers using a weight matrix, a nonlinear relation (or a linear relation) using an activation function in each layer, and a bias. Various functions such as a logistic sigmoid function (logistic function), a hyperbolic tangent function, a rectified linear function, a linear map, an identity map, and a maxout function can be selected as the activation function according to a purpose.

The weight matrix and the bias are parameters (hereinafter referred to as model parameters) defining an operation of the multilayer network. The composite function with parameters changes its form as a function depending on the selection of the model parameters. In the multilayer network, a function capable of outputting a preferable result from its output layer can be defined by appropriately setting the constituent model parameters.

The model parameters are set by executing learning using the learning data and an error function. The error function is a function representing an approximation between the output from the multilayer network to which the biological parameters are inputted, and the training data. Typical examples of the error function include a squared error function, a maximum likelihood estimation function, and a cross entropy function. A function selected as the error function depends on a problem dealt with by the multilayer network (e.g., a regression problem, a binary classification problem, and a multi-class classification problem). For example, a value minimizing the error function is determined as the model parameters during a creation process of the model M1.

Based on the knowledge base 21 stored in the knowledge base storage apparatus 20 and the model M1 stored in the storage 103, the comparison function 112 compares the biological parameter condition values related to the derivation of the medical knowledge derived from the knowledge base 21 and the derivation of the inference result derived from the model M1, for each item of the biological parameters.

More specifically, the comparison function 112 compares the biological parameter conditions on which the medical knowledge derived from the knowledge base 21 and the inference result derived from the model M1 indicate an identical matter or related matters, for each item of the biological parameters.

For example, when both of the medical knowledge derived from the knowledge base 21 and the inference result derived from the model M1 indicate cardiac insufficiency symptoms, the comparison function 112 determines that the identical matter is derived from the medical knowledge and the inference result. In this case, the comparison function 112 compares the biological parameter conditions related to the medical knowledge and the inference result from which the “cardiac insufficiency” is derived, for each item of the biological parameters. For example, when the medical knowledge derived from the knowledge base 21 indicates cardiomyopathy symptoms and the inference result derived from the model M1 indicates cardiac insufficiency symptoms, the comparison function 112 determines that the related matters are derived from the medical knowledge and the inference result. In this case, the comparison function 112 compares the biological parameter conditions related to the medical knowledge and the inference result from which the related matters are derived, for each item of the biological parameters.

More specifically, based on the medical knowledge prescribed in the knowledge base 21, the comparison function 112 acquires, from the model M1, the biological parameter conditions (the items of the biological parameters and their condition values) related to the derivation of the inference result indicating the identical or related matter with that of the medical knowledge. The comparison function 112 compares the biological parameter condition values acquired from the model M1 with the biological parameter condition values prescribed in the knowledge base 21, for each item of the biological parameters, and outputs the comparison results to the error calculation function 113.

Note that any criterion for determination can be set for determining the identical or related matter. Additionally, a set of the medical knowledge and the inference result to be compared may be instructed by a manual operation via the input interface 101.

Moreover, various methods can be used as a method for acquiring the biological parameter condition values from the model M1. The comparison function 112 may acquire the items of the biological parameters and their condition values contributing to the derivation of the inference result from the model M1 by using, for example, a known technique such as feature importance measurement. The comparison function 112 may also measure contributions to the derivation of the inference result as importance for each item of the biological parameters and set a threshold or the like to select the biological parameter item having a high contribution.

Furthermore, the biological parameter condition values acquired from the model M1 by the comparison function 112 are not limited to quantitative values but may be qualitative. For example, the comparison function 112 may acquire chronological change tendencies (e.g., an increase and a decrease) of the biological parameters as the condition values. Note that the forms of the condition values acquired from the model M1 preferably match the forms of the condition values of the corresponding biological parameter items prescribed in the knowledge base 21.

The error calculation function 113 calculates a deviation degree between the biological parameter conditions of the knowledge base 21 and the model M1 based on the comparison results of the comparison function 112.

More specifically, the error calculation function 113 determines whether each biological parameter item has a deviation based on a difference between two condition values to be compared, or a difference in positive/negative coefficients. The error calculation function 113 calculates an error with a penalty based on the number of the biological parameters determined to have a deviation or the condition values thereof.

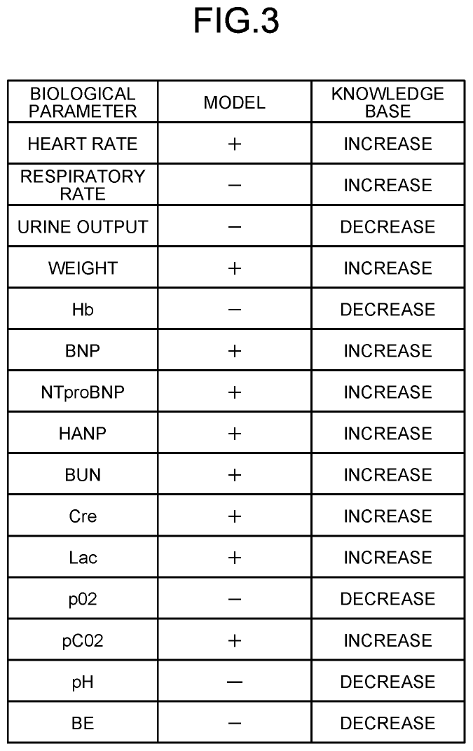

An operation of the error calculation function 113 will now be described with reference to FIG. 3. FIG. 3 is a schematic view illustrating an example of the biological parameter comparison results.

FIG. 3 illustrates the biological parameter conditions related to the prediction of cardiac insufficiency symptoms acquired from the knowledge base 21 and the model M1. More specifically, 15 items including heart rate, respiratory rate, and urine output are cited as the biological parameter items common between the knowledge base 21 and the model M1 in FIG. 3. FIG. 3 illustrates an example in which chronological increasing/decreasing tendencies are acquired as the biological parameter condition values in the knowledge base 21. FIG. 3 also illustrates an example in which an increasing/decreasing tendency of each biological parameter acquired from the model M1 is represented by a positive/negative (+, −) coefficient.

For example, for the biological parameter “heart rate”, it is understood that both of the knowledge base 21 and the model M1 have increasing tendencies (+), which indicates that the cardiac insufficiency symptoms are predicted. In this case, the error calculation function 113 determines that the parameter “heart rate” has “no deviation”. Meanwhile, for the parameter “respiratory rate”, it is understood that the increasing/decreasing tendencies of the knowledge base 21 and the model M1 are in reverse relation. In this case, the error calculation function 113 determines that the parameter “respiratory rate” has “a deviation”.

If there is any biological parameter determined to have “a deviation” in the comparison results of the comparison function 112, the error calculation function 113 calculates an error function L′ with a penalty reflecting a degree of deviation (hereinafter referred to as deviation degree) between the biological parameter conditions of the knowledge base 21 and the model M1 based on the following equation (1). Note that the error with a penalty corresponds to an output value of the error function L′ with a penalty.

L′=L+λ×R (1)

In the above equation (1), “L” is an error function set in the initial creation of the model M1. “λ” is a hyperparameter of the model M1, for which any constant can be set. “R” is a term determined according to the determination results of the error calculation function 113 (hereinafter referred to as R term). A penalty term is formed by the A and the R term.

The R term is represented by, for example, a polynomial composed of a weighting factor in an input value to a node of each layer constituting the model M1. More specifically, the R term is set so as to increase the error function L′ with the penalty as the deviation degree is larger.

For example, based on the number of the biological parameters determined to have a deviation by the error calculation function 113, the R term is set so as to increase the error function L′ with the penalty as the number is larger. This error function L′ with the penalty is used to correct the model M1 so as to minimize the L′, thereby adjusting the operation of the model M1. This allows the biological parameter conditions used for the derivation of the inference result by the model M1 to effectively approach the biological parameter conditions prescribed in the knowledge base 21.

In FIG. 3, the biological parameter positive/negative coefficients of which are in reverse relation is determined to have a deviation. However, the determination method is not limited thereto. For example, when the medical data condition values are quantitatively represented, the error calculation function 113 may determine that the biological parameter in which a difference between the condition values exceeds a threshold has a deviation. In this case, based on the difference between the condition values determined to have a deviation by the error calculation function 113, the R term is set so as to increase the error function L′ with the penalty as the difference is larger.

For example, based on a square sum value of the differences between the condition values, the R term may be set so as to increase the error function L′ with the penalty as the value is larger. This error function L′ with the penalty is used to adjust the model M1. This allows the biological parameter conditions used for the derivation of the inference result by the model M1 to effectively approach the biological parameter conditions prescribed in the knowledge base 21.

While the example in which the presence of deviation is determined for each item of the biological parameters has been described using FIG. 3, a plurality of biological parameter items may be grouped together, and the presence of deviation may be determined for each group. For example, for a disease such as a heart disease, a plurality of biological parameters can have a significant relation. In this case, the error calculation function 113 puts the biological parameters having a significant relation into one group, and determines that there is a deviation when any or all of the condition values in the group differ between the knowledge base 21 and the model M1.

The error calculation function 113 can thereby determine the deviation degree of the biological parameter conditions between the knowledge base 21 and the model M1, for each group of the biological parameters. Consequently, the error calculation function 113 can determine the presence of deviation based on the relation among the biological parameters such as the biological parameters having a significant relation.

Moreover, while the example in which the same biological parameter items are compared between the knowledge base 21 and the model M1 has been described using FIG. 3, the biological parameter items acquired from the model M1 and the biological parameter items prescribed in the knowledge base 21 possibly disagree with each other. For example, when the number of the biological parameter items contributing to the inference of the model M1 exceeds the number of the biological parameter items prescribed in the knowledge base 21, the model M1 derives the inference result by using other biological parameter items than those prescribed in the knowledge base 21 as well.

In such a case, the error calculation function 113 may calculate the error function L′ with the penalty the R term of which is set so as to decrease the contributions of the other biological parameter items. The error calculation function 113 thereby allows the biological parameter conditions used for the inference of the model M1 to approach the biological parameter conditions prescribed in the knowledge base 21.

The learning function 111 adjusts the operation of the model M1 based on the determination results of the error calculation function 113. More specifically, the learning function 111 adjusts the model parameters of the model M1 in a direction to decrease the error with the penalty as the output value of the error function L′ with the penalty based on the error function L′ with the penalty calculated by the error calculation function 113. For example, the learning function 111 adjusts the model parameters of the model M1 in the direction to decrease the error with the penalty by giving feedback to the model parameters of the model M1 by, for example, a backpropagation method based on the error function L′ with the penalty.

The learning function 111 can obtain the model parameters that minimize the deviation between the biological parameter conditions of the knowledge base 21 and the model M1 by repeated learning using many pieces of the verification data. The learning function 111 creates the model M1 matching the conditions in the knowledge base 21 as described above.

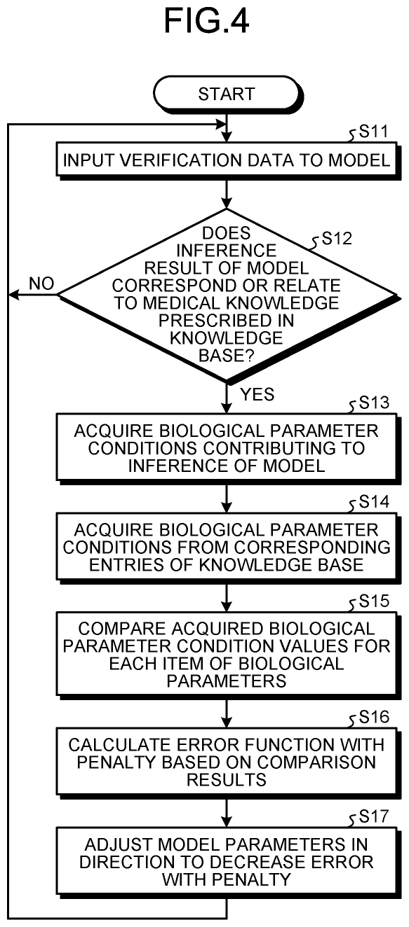

Next, the processing performed by the learning assistance apparatus 10 will be described with reference to FIG. 4. FIG. 4 is a flowchart illustrating an example of the processing performed by the learning assistance apparatus 10. As a premise of the processing, the model M1 created based on the learning data is stored in the storage 103.

First, the learning function 111 inputs the verification data (the clinical data 31) to the model M1 (step S11). In response to the input of the clinical data, the model M1 derives the inference result such as disease diagnoses, therapeutic effect determination, and prognosis prediction based on the biological parameter conditions included in the verification data.

The comparison function 112 determines whether the inference result of the model M1 corresponds or relates to the medical knowledge prescribed in the knowledge base 21 by referring to the knowledge base 21 (step S12). If determining that there is no correspondence or relation, (No at the step S12), the comparison function 112 returns the processing to the step S11.

On the other hand, if determining that the inference result corresponds or relates to the medical knowledge (Yes at the step S12), the comparison function 112 acquires the biological parameter conditions contributing to the inference of the inference result from the model M1 (step S13). The comparison function 112 also acquires the biological parameter conditions related to the derivation of the medical knowledge from the corresponding entries of the knowledge base 21 (step S14).

The comparison function 112 compares the biological parameter condition values acquired at the steps S13 and S14 for each item of the biological parameters, and outputs the comparison results to the error calculation function 113 (step S15).

Subsequently, the error calculation function 113 calculates the error function L′ with the penalty based on the comparison results at the step S15 (step S16). The learning function 111 adjusts the model parameters of the model M1 in the direction to decrease the error with the penalty of the error function L′ with the penalty calculated at the step S16 (step S17), and returns the processing to the step S11.

As described above, the learning assistance apparatus 10 compares the medical data conditions related to the derivation of the medical knowledge and the derivation of the inference result for each item of the biological parameters based on the knowledge base 21 and the model M1, and outputs the comparison results. The learning assistance apparatus 10 then calculates the error function L′ with the penalty representing the deviation degree between the biological parameter conditions related to the derivation of the medical knowledge and the derivation of the inference result, and adjusts the model parameters of the model M1 in the direction to decrease the error with the penalty.

The learning assistance apparatus 10 can thereby obtain the model M1 in which the deviation between the biological parameter conditions of the knowledge base 21 and the model M1 is decreased. Consequently, the learning assistance apparatus 10 can assist the creation of the model M1 matching the conditions in the knowledge base 21.

Note that the above embodiment can be appropriately modified and implemented by partially changing the configuration or functions of the learning assistance apparatus 10. Hereinafter, some modifications related to the above embodiment will be described as other embodiments. Note that different points from those of the above embodiment will be mainly described below, and a detailed description on points common with the above description will be omitted. Additionally, the modifications described below may be implemented individually, or may be appropriately combined together and implemented.

### First Modification

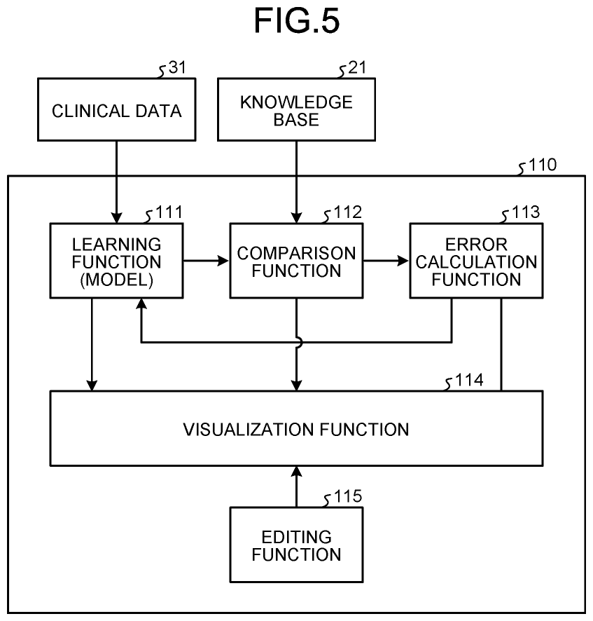

FIG. 5 is a diagram illustrating a function configuration example of the processing circuitry 110 according to the present modification. As illustrated in FIG. 5, the processing circuitry 110 includes a visualization function 114 and an editing function 115 in addition to the respective functions described using FIG. 2. The visualization function 114 is an example of an output unit and a visualization unit. The editing function 115 is an example of an editing unit. Note that the comparison function 112 of the present modification outputs the comparison results of the biological parameters to the error calculation function 113 and the visualization function 114.

The visualization function 114 displays (outputs) a screen visualizing the processing results or processing states of the learning function 111, the comparison function 112, and the error calculation function 113 on the display 102.

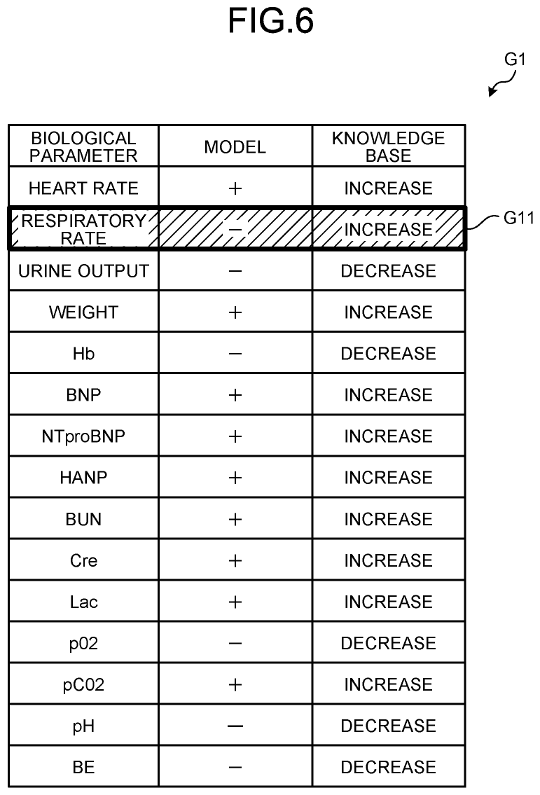

For example, the visualization function 114 displays a screen visualizing the comparison results of the comparison function 112 on the display 102. As an example, the visualization function 114 displays a screen G1 visualizing the comparison results of the comparison function 112 for each condition of the medical data (the biological parameters) on the display 102 as illustrated in FIG. 6. FIG. 6 is a view illustrating an example of the screen G1 displayed by the visualization function 114. The comparison results described using FIG. 3 are visualized in FIG. 6.

The visualization function 114 may also highlight the biological parameter determined to have a deviation by using the determination results of the error calculation function 113. FIG. 6 illustrates an example in which the visualization function 114 highlights an entry G11 of the biological parameter (respiratory rate) determined to have a deviation.

An operator of the learning assistance apparatus 10 can thereby easily check a difference in the biological parameter condition between the knowledge base 21 and the model M1 by looking at the screen G1 displayed by the visualization function 114. Consequently, the learning assistance apparatus 10 can assist the creation of the model M1 matching the conditions in the knowledge base 21.

The visualization function 114 also visualizes and displays on the display 102 the error function L′ with the penalty calculated by the error calculation function 113. For example, the visualization function 114 displays the error function L′ with the penalty in a state editable by the editing function 115. The operator of the learning assistance apparatus 10 can thereby easily check the contents of the error function L′ with the penalty calculated by the error calculation function 113. Consequently, the learning assistance apparatus 10 can improve convenience in creating the model M1, and can assist the creation of the model M1 matching the conditions in the knowledge base 21.

The editing function 115 receives an editing operation for the processing results or processing states of the learning function 111, the comparison function 112, and the error calculation function 113 via the input interface 101.

For example, the editing function 115 receives an editing operation for the determination results of the error calculation function 113 displayed on the display 102. As an example, the editing function 115 receives an operation to instruct the biological parameter to be incorporated into or excluded from the penalty term (R term) based on a screen showing the determination results. In this case, the error calculation function 113 executes a process of incorporating the instructed biological parameter into the penalty term or excluding the instructed biological parameter from the penalty term via the visualization function 114. The learning assistance apparatus 10 enables the operator of the learning assistance apparatus 10 to perform the editing operation for the error function L′ with the penalty in such a manner. Consequently, the learning assistance apparatus 10 can assist the creation of the model M1 matching the conditions in the knowledge base 21.

For example, the editing function 115 also receives an editing operation for the error function L′ with the penalty displayed on the display 102. As an example, the editing function 115 receives an editing operation for the hyperparameter “A” of the error function L′ with the penalty. In this case, the error calculation function 113 executes a process of changing the hyperparameter value via the visualization function 114. The learning assistance apparatus 10 enables the operator of the learning assistance apparatus 10 to perform the editing operation for the error function L′ with the penalty in such a manner. Consequently, the learning assistance apparatus 10 can assist the creation of the model M1 matching the conditions in the knowledge base 21.

While the configuration example in which the editing operation received by the editing function 115 is transmitted to the error calculation function 113 via the visualization function 114 has been described using FIG. 6, the modification is not limited to this example. The editing operation received by the editing function 115 may be directly transmitted to the error calculation function 113.

When the error function L′ with the penalty is edited, the learning function 111 may create one model M1 based on the edited error function L′ with the penalty, or may create models M1 individually based on the error functions L′ with the penalty before and after editing. In the latter case, the learning function 111 creates the models M1 individually based on the error functions L′ with the penalty before and after editing, and stores the models M1 of different generations in the storage 103. Moreover, when the models M1 of different generations are stored, the learning function 111, the comparison function 112, the error calculation function 113, and the visualization function 114 may perform the following processing.

First, the learning function 111 calculates a matching rate (correct answer rate) between the inference result of the model M1 and the training data for each generation of the models M1 by using the verification data. The comparison function 112 and the error calculation function 113 calculate a deviation rate (or a matching rate) between the biological parameter conditions contributing to the inference of the model M1 and the biological parameter conditions prescribed in the knowledge base 21, for each generation of the models M1. Any method may be employed for calculating the deviation rate. For example, the percentage of the biological parameters determined to have a deviation in the biological parameters acquired from the knowledge base 21 and the model M1 may be calculated as the deviation rate.

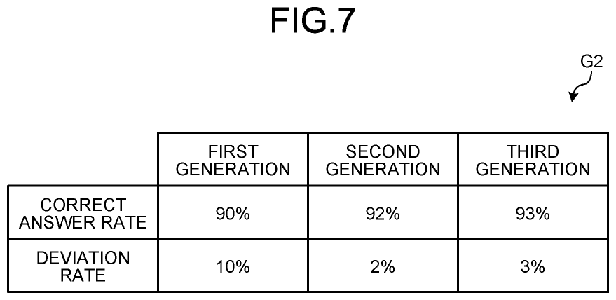

The visualization function 114 displays, on the display 102, the information regarding the models M1 of the respective generations calculated by the learning function 111, the comparison function 112, and the error calculation function 113 in a comparable state. For example, the visualization function 114 displays a screen G2 showing the correct answer rates of the models M1 and the deviation rates from the knowledge base 21 on the display 102 for each generation of the models M1 as illustrated in FIG. 7.

FIG. 7 is a view illustrating an example of the screen G2 displayed by the visualization function 114. FIG. 7 illustrates an example of displaying the correct answer rates and the deviation rates of the models M1 of three generations. The first generation means the model M1 based on the error function L′ with the penalty that is automatically set by the error calculation function 113. The second generation corresponds to the model M1 after editing the error function L′ with the penalty of the first generation, and the third generation to the model M1 after further editing the error function L′ with the penalty of the second generation.

As illustrated in FIG. 7, with regard to, for example, the deviation rate, it is understood that the model M1 of the second generation has a highest matching rate with the knowledge base 21. Additionally, with regard to, for example, the correct answer rate, it is understood that the model M1 of the third generation has a highest correct answer rate. The screen G2 provided by the visualization function 114 thereby enables the operator of the learning assistance apparatus 10 to easily compare the performances (evaluation values) of the models M1 of the respective generations. As described above, the learning assistance apparatus 10 can propose the models M1 in the states before and after editing the error function L′ with the penalty to the operator. Consequently, the learning assistance apparatus 10 can assist the creation of the model M1 matching the conditions in the knowledge base 21.

### Second Modification

While the example in which the knowledge base storage apparatus 20 holds the knowledge base 21 has been described in the above embodiment, the learning assistance apparatus 10 may hold the knowledge base 21. While the example in which the clinical data storage apparatus 30 holds the clinical data 31 has been described in the above embodiment, the learning assistance apparatus 10 may hold the clinical data 31.

Moreover, while the example in which the learning assistance apparatus 10 creates the model M1 and the model M1 is stored in the storage 103 has been described in the above embodiment, the embodiment is not limited to this example. For instance, the model M1 may be created by an external apparatus other than the learning assistance apparatus 10, or may be stored in an external apparatus accessible by the learning assistance apparatus 10.

While the example in which the function configuration of the learning assistance apparatus 10 is achieved by the processing circuitry 110 has been described in the above embodiment, the embodiment is not limited to this example. For instance, the function configuration in the present specification may be achieved using only hardware or a combination of hardware and software.

The term “processor” used in the above description means, for example, a central processing unit (CPU), a graphics processing unit (GPU), or a circuit such as an application specific integrated circuit (ASIC) and a programmable logic device (e.g., a simple programmable logic device (SPLD), a complex programmable logic device (CPLD), and a field programmable gate array (FPGA)). For example, when the processor is a CPU, the processor achieves the functions by reading and executing the computer programs stored in the storage 103. For example, when the processor is an ASIC, the functions are directly incorporated into the circuit of the processor as a logic circuit instead of storing the computer programs in the storage 103. The respective processors of the present embodiment do not necessarily have to be each configured as a single circuit, but may be configured as one processor composed of a plurality of independent circuits so as to achieve the functions. Moreover, a plurality of constituent elements in the respective drawings may be integrated into one processor to achieve the functions.

The computer programs executed by the processor are provided by being previously installed in a read only memory (ROM) or a storage. Note that the computer programs may be provided by being recorded in a computer-readable storage medium such as a compact disc (CD)-ROM, a flexible disk (FD), CD-recordable (R), and a digital versatile disc (DVD) in the form of files installable or executable in the apparatuses. The computer programs may be stored in a computer connected to a network such as the Internet and may be provided or distributed by being downloaded via the network. For example, the computer programs are configured by modules including the above respective functional units. In the actual hardware, the CPU reads and executes the computer programs from a storage medium such as a ROM such that each module is loaded in a primary storage and generated in the primary storage.

According to at least one of the embodiments described above, the creation of the model matching the conditions in the knowledge base can be assisted.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A learning assistance apparatus comprising a processing circuitry configured to
compare, based on a knowledge base in which a condition of medical data as an index for medical judgment and medical knowledge derived from the condition are associated with each other, and a model designed to derive a medical inference result from a condition of medical data concerning a subject in response to input of the medical data, the medical data condition related to the derivation of the medical knowledge and the medical data condition related to the derivation of the inference result, for each item of the medical data, and
output a result of the comparison.

2. The learning assistance apparatus according to claim 1, wherein
the processing circuitry compares the medical data conditions on which the medical knowledge and the inference result indicate an identical matter or related matters, for each item of the medical data.

3. The learning assistance apparatus according to claim 1, wherein
the processing circuitry displays a screen visualizing the comparison result for each condition of the medical data.

4. The learning assistance apparatus according to claim 1, wherein
the processing circuitry adjusts an operation of the model based on the comparison result.

5. The learning assistance apparatus according to claim 4, wherein
the processing circuitry calculates an error function reflecting a deviation degree between the medical data conditions related to the derivation of the medical knowledge and the derivation of the inference result based on the comparison result, and
adjusts a parameter related to the operation of the model based on the calculated error function.

6. The learning assistance apparatus according to claim 5, wherein
the processing circuitry calculates the error function based on a difference between condition values of the medical data between the medical knowledge and the inference result.

7. The learning assistance apparatus according to claim 5, wherein
the processing circuitry calculates the error function based on the number of the medical data positive/negative coefficients of which are reversed between the medical knowledge and the inference result.

8. The learning assistance apparatus according to claim 5, wherein
the processing circuitry edits the error function according to an editing operation received via an input interface.

9. The learning assistance apparatus according to claim 1, wherein
the processing circuitry uses, as the knowledge base, a guideline created from a medical viewpoint and prescribing a relation between the medical data condition and the medical knowledge.

10. A learning assistance method comprising
comparing, based on a knowledge base in which a condition of medical data as an index for medical judgment and medical knowledge derived from the condition are associated with each other, and a model designed to derive a medical inference result from a condition of medical data concerning a subject in response to input of the medical data, the medical data condition related to the derivation of the medical knowledge and the medical data condition related to the derivation of the inference result, for each item of the medical data, and
outputting a result of the comparison.
